(19) United States
(12) Patent Application Publication
(10) Pub. No.: US 2009/0053195 A1
(43) Pub. Date: Feb. 26, 2009

(54) BIOMARKERS FOR ACUTE GRAFT REJECTION

(76) Inventors: Friedrich Raulf, Freiburg (DE); Lukas Roth, Riehen (CH); Johannes Voshol, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/574,039

(22) PCT Filed: Sep. 5, 2005
(86) PCT No.: PCT/EP05/09526

§ 371 Date: Feb. 21, 2007

(30) Foreign Application Priority Data
Sep. 6, 2004 (GB) 0419728.1

(51) Int. Cl. C12Q 1/68; C12Q 1/00; G01N 33/53; A61K 39/395; C07K 16/00; A61P 37/06

(52) U.S. Cl. 424/130.1; 435/6; 435/4; 435/7.1; 530/387.1

(57) ABSTRACT

Methods are disclosed for the early non-invasive diagnosing of acute rejection (AR) in a transplanted subject, monitoring AR in a transplanted subject at risk of developing AR, preventing, inhibiting, reducing or treating AR in a transplanted subject, or identifying agents for use in the prevention, inhibition, reduction or treatment of AR, based on genes which are differentially expressed in peripheral blood or transplant biopsy tissues, before full clinical manifestation of AR is detected in the transplanted subject.

The present invention relates to a method of monitoring the status of a transplanted tissue or organ in a recipient. In particular, the invention relates to the use of gene expression analysis to determine early non-invasive diagnosis of acute allograft rejection.

Although the one-year graft survival after kidney transplantation is around 90% due to immunosuppressives like cyclosporine A (Neoral®), both, acute and chronic rejection are still important processes as causes for graft loss. The early detection of acute renal allograft rejection by a non-invasive method is an unmet medical need since today only a biopsy can confirm an acute rejection episode. The current gold standard diagnostic assay for acute rejection (AR) is the measurement of serum creatinine (sCrea), but the rise of sCrea is a late event when about 70% of the kidney graft is already damaged, and it is quite unspecific needing a final confirmation by a diagnostic biopsy.

There is a need to have non-invasive acute rejection biomarkers that are predictable, sensitive, reliable, and easily measurable. Such an aid would be valuable e.g. for sparing biopsies and therefore increasing the quality of life for the transplant patient, for the optimization of drug treatment adjustments by the earlier detection of AR episodes and finally by improving long-term graft survival that was shown to be closely correlated to the number and severity of AR episodes.

The present invention relates to the identification of genes which are differentially expressed in peripheral blood as well as in transplant biopsies, e.g. renal biopsies, in patients who are developing AR. The resulting gene expression pattern of a subset of the genes allows a highly statistically significant predictability of the occurrence of AR. For example, the genes identified as mRNA transcripts in the blood of a rat life-supporting kidney allotransplantation model (BN-to-Lewis) 3, 4, and 5 days before AR at day 7 occurred, are indicated in Table 1. The NCBI RefSeq or GenBank accession numbers of the 28 genes disclosed in this application are listed in Table 1 and can be retrieved from NCBI. The sequences as shown under the corresponding NCBI GenBank/RefSeq accession number are incorporated herein by reference.

In addition protein markers, e.g. as listed in Table 4, have been identified in serum of rats with and without AR of renal grafts.

The genes identified according to the invention are useful predictive biomarkers for the early diagnosis of AR in transplanted subjects. Any selection, of at least one, of these genes can be utilized as surrogate biomarker for early diagnosis of AR. In particularly useful embodiments, a plurality of these genes can be selected and their mRNA expression monitored in patient blood simultaneously to provide expression profiles for use in various aspects.

Accordingly, the invention provides the use of a gene, e.g. as listed in Table 1 as an early biomarker for acute transplant rejection, e.g. as a biomarker for AR before any overt clinical or histological manifestation.

In a further embodiment, the levels of the gene expression products (proteins) can be monitored in various body fluids, including, but not limited to, blood plasma, serum, lymph, urine, stool and bile, or in biopsy tissues. This expression product level can be used as surrogate markers for early diagnosis of AR and can provide indices of therapy responsiveness. An example is e.g. the protein encoded by the Pre-B-cell colony-enhancing factor (NCBI RefSeq accession number NM_{—}005746) or at least one of the protein of Table 4.

Methods of detecting the level of expression of mRNA are well-known in the art and include, but are not limited to, reverse transcription PCR, real time quantitative PCR, Northern blotting and other hybridization methods.

Accordingly, the invention provides the use of an expression product of (e.g. a protein encoded by) a gene as listed in Table 1 and/or a protein as listed in Table 4 as an early biomarker for acute transplant rejection, e.g. as a biomarker for AR before any overt clinical or histological manifestation.

A particularly useful method for detecting the level of mRNA transcripts obtained from a plurality of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the blood or biopsy obtained from the transplanted subject at risk of developing AR can be compared with the gene expression profile derived from the sample obtained from a transplanted subject that is not developing AR.

In a further embodiment, measuring expression profiles of one or a plurality of these genes or encoded proteins can provide valuable molecular tools for examining the efficacy of drugs for inhibiting, e.g. preventing or treating, AR. Changes in the expression profile from a baseline profile while the transplanted patient is exposed to therapy. Accordingly, this invention also provides a method for screening a transplanted subject to determine the likelihood that the subject will respond to the AR therapy, methods for the identification of agents that are useful in treating a transplanted subject having AR signs and methods for monitoring the efficacy of certain drug treatments for AR.

In one embodiment, the invention provides a method of identifying at least one gene and/or one protein which is differentially expressed in peripheral blood in a transplanted subject prior to the onset of AR by
i) determining the level of mRNA expression corresponding to the gene at different time points and using a reference as baseline; and/or
ii) determining the level of protein expression corresponding to the gene at different time points using a reference as baseline; and
iii) analyzing said expression level data using filtering on fold changes and/or statistical tests.

The term “differentially expressed” refers to a given allograft gene expression level and is defined as an amount which is substantially greater or less than the amount of the corresponding baseline expression level.

A reference in (ii) or (iii) may be e.g. one or more subjects known not to develop AR.

In another aspect, the invention provides a method of early diagnosing AR in a transplanted subject, e.g. in vitro, by detecting a differentially expressed gene in a given body fluid or allograft tissue sample. For example, the method may comprise

a) taking as a baseline value the level of mRNA expression corresponding to or protein encoded by at least one gene in a body fluid, e.g. in peripheral blood, the gene originating from a preclinical AR model, e.g. a gene as identified in Table 1;
b) detecting a level of mRNA expression corresponding to or protein encoded by the at least one gene in a body fluid, e.g. in peripheral blood, of a transplant patient on a regular basis, e.g. every 4 weeks;
c) comparing the first value with the second value, wherein a first value lower or higher than the second value predicts that the transplanted subject is developing AR.

By prior to the onset of AR or early diagnosis of AR is meant before any overt clinical or histological manifestation of AR is detected in the transplanted subject.

In another aspect, the invention provides a method for monitoring, e.g. preventing or inhibiting or reducing or treating AR in a transplanted subject at risk of developing AR with an AR inhibitor, e.g. a small molecule, an antibody or other therapeutic agent or candidate agent. Monitoring the influence of agents, e.g. drug compounds, on the level of expression of a marker of the invention can be applied not only in basic drug screening, but also in clinical trials. For example, the effectiveness of an agent to affect marker expression can be monitored in clinical trials of transplanted subjects receiving treatment for the inhibition of AR.

Such a method comprises:

a) obtaining a pre-administration sample from a transplanted subject prior to administration of the agent,
b) detecting the level of expression of mRNA corresponding to or protein encoded by the at least one gene in the pre-administration sample,
c) obtaining one or more post-administration samples from the transplanted patient,
d) detecting the level of expression of mRNA corresponding to or protein encoded by the at least one gene in the post-administration sample or samples,
e) comparing the level of expression of mRNA or protein encoded by the at least one gene in the pre-administration sample with the level of expression of mRNA or protein encoded by the at least one gene in the post-administration sample or samples, and
f) adjusting the agent accordingly.

For example, increased or decreased administration of the agent may be desirable to change the level of expression of the at least one gene to higher or lower levels than detected. In above method, the agent can also be administered alone or in combination with other agents in a combined therapy, preferably with immunosuppressive agents and/or agents effective in AR.

Accordingly, incorporation of gene expression profiling data from human body fluids, e.g. peripheral blood, or biopsies, e.g. human renal protocol biopsies, will help improve the patient selection process during clinical trials aimed at both treatment and prevention of the progression towards AR.

In a yet other aspect, the invention further provides a method for identifying agents for use in the prevention, inhibition, reduction or treatment of AR comprising monitoring the level of mRNA expression of at least one gene or protein encoded as disclosed above.

In a further aspect, the invention provides a method for preventing, inhibiting, reducing or treating AR in a transplant subject in need of such treatment comprising administering to the subject a compound that modulates the synthesis, expression or activity of one or more genes or gene expression products, e.g. as disclosed in Tables 1 and/or 4, so that at least one symptom of AR is ameliorated.

In a further aspect, the invention provides a compound, e.g. a small molecule, an antibody or other therapeutic agent or candidate agent, which modulates the synthesis, expression of activity of one or more genes or gene expression products identified above, e.g. as indicated in Tables 1 and/or 4, for use as a medicament, e.g. for the prevention or treatment of AR in a transplanted subject.

In a further aspect, the invention provides the use of a compound, e.g. a small molecule, an antibody or other therapeutic agent or candidate agent, which modulates the synthesis, expression of activity of one or more genes or gene expression products identified above, e.g. in Tables 1 and/or 4, for prevention or treatment of AR in a transplanted subject.

In a further aspect, the invention provides the use of a compound, e.g. a small molecule, an antibody or other therapeutic agent or candidate agent, which modulates the synthesis, expression of activity of one or more genes or gene expression products identified above, e.g. in Tables 1 and/or 4, for the preparation of a medicament for prevention or treatment of AR in a transplanted subject.

By transplanted subject is meant a subject receiving tissue or organ from a donor, preferably from the same species, e.g. kidney, heart, lung, combined heart and lung, liver, pancreas, bowel (e.g., colon, small intestine, duodenum), neuronal tissue, pancreatic islets, limbs.

Preferably more than one gene, e.g. a set of genes, is used in the methods of the invention.

As already mentioned any selection, of at least one, of the genes indicated in Table 1 and/or of the gene expression products, e.g. as indicated in Table 4 can be used.

Gene expression profiles can be generated using e.g. the Affymetrix microarray technology. Microarrays are known in the art and consist of a surface to which probes that correspond in sequence to gene products (e.g. mRNAs, polypeptides, fragments thereof etc.). can be specifically hybridized or bound to a known position. Hybridization intensity data detected by the scanner are automatically acquired and processed by the Affymetrix Microarray Suite (MAS5) software. Raw data is normalized to expression levels using a target intensity of 150.

An alternate and preferred method to measure gene expression profiles of a small number of different genes is by e.g. either classical TaqMan® Gene Expression Assays or TaqMan® Low Density Array—micro fluidic cards (Applied Biosystems). Here, quantitative data are obtained by real-time RT-PCR in a small reaction volume.

The transcriptional state of a cell may be measured by other gene expression technologies known in the art. Several such technologies produce pools of restriction fragments of limited complexity for electrophoretic analysis, such as methods combining double restriction enzyme digestion with phasing primers (e.g. EP-A1-0 534858), or methods selecting restriction fragments with sites closest to a defined mRNA end (e.g. Prashar et al; Proc. Nat. Acad. Sci., 93, 659-663, 1996). Other methods statistically sample cDNA pools, such as by sequencing sufficient bases (e.g. 20-50 bases) in each multiple cDNAs to identify each cDNA, or by sequencing short tags (e.g. 9-10 bases) which are generated at known positions relative to a defined mRNA end (e.g. Velculescu, Science, 270, 484-487, 1995) pathway pattern.

In another embodiment of the present invention, a protein corresponding to a marker is detected. A preferred agent for detecting a protein of the invention is e.g. an antibody capable of binding to the protein, preferably an antibody with a detectable label. Antibodies can be polyclonal, or preferably, monoclonal. An intact antibody or a fragment thereof (e.g. Fab or F(ab′)₂ can be used. The term “labeled” is intended to encompass direct labeling of the antibody by coupling a detectable substance to antibody, as well as indirect labeling of the antibody by reactivity with another reagent that is directly labeled. A variety of formats can be employed to determine whether a sample contains a protein that binds to a given antibody. Examples of such formats include e.g. enzyme immunoassay, radioimmunoassay, Western blot analysis and ELISA.

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with main memory. The external components include mass data storage. This mass storage can be one or more hard disks. Other external components include user interface device, which can be a monitor and keyboards, together with pointing device or other graphic input devices. Typically, the computer system is also linked to other local computer systems, remote computer systems or wide area communication networks, e.g. Internet. This network link allows the computer system to share data and processing tasks with other computer systems.

Loaded into memory during operation of this system are several software components which are both standard in the art and special to the instant invention. These software components collectively cause the computer system to function according to the methods of this invention. These software components are typically stored on mass storage or on removable media, e.g. floppy disks or CD-ROM. The software component represents the operating system, which is responsible for managing the computer system and its network interconnections. Preferably, the methods of this invention are programmed in mathematical software packages, which allow symbolic entry of equations and high-level specification of processing, including algorithms to be used, and thereby freeing a user of the need to procedurally program individual equations or algorithms.

In preferred embodiments, the analytic software component actually comprises separate software components that interact with each other. Analytic software represents a database containing all data necessary for the operation of the system. Such data will generally include, but is not limited to, results of prior experiments, genome data, experimental procedures and cost, and other information, which will be apparent to those skilled in the art. Analytic software includes a data reduction and computation component comprising one or more programs which execute the analytic methods of the invention. Analytic software also includes a user interface which provides a user of the computer system with control and input of test network models and, optionally, experimental data. The user interface may comprise a drag-and-drop interface for specifying hypotheses to the system. The user interface may also comprise means for loading experimental data from the mass storage component, from removable media or from a different computer system communicating with the instant system over a network.

The invention also provides a process for preparing a database comprising at least one of the markers set forth in this invention, e.g. mRNAs. For example, the polynucleotide sequences are stored in a digital storage medium such that a data processing system for standardized representation of the genes that identify early diagnosis of AR. The data processing system is useful to analyze gene expression between two body fluid or tissue samples taken at different time point, e.g. at the transplantation day and post-transplantation. The isolated polynucleotides are sequenced. The sequences from the samples may be compared with the sequence(s) present in the database using homology search techniques. Alternative computer systems and methods for implementing the analytic methods of this invention will be apparent to one skilled in the art and are intended to be comprehended within the accompanying claims.

### EXAMPLES

### Preclinical AR Model for the Identification of Diagnostic Markers

For a biomarker study in the life-supporting BN-to-Lewis rat kidney transplantation model (occurrence of AR at day 7), a total of 10 allograft and 10 isograft rats each are transplanted in parallel without any immunosuppressive regimen and terminated at three different time points (3, 4, 5 days post transplantation). In addition, two groups with 6 rats each for non life-supporting isograft controls were performed. About 1360 probes (blood, serum, urine, graft tissue) are sampled in a concerted effort. Measurements of graft histology and blood chemistry changes are applied along with combined proteomic, transcriptomic, and metabonomic analyses.

RNA extraction: Whole blood is collected into EDTA-ET tubes (Sarstedt) and 2 aliquots (2 ml, 0.25 ml) are transferred into a 15 ml Falcon or a 2 ml Eppendorf tube, respectively, filled each with the same volume of PBS without Ca²⁺/Mg²⁺. After mixing, an equal volume Nucleic Acid Purification Lysis Solution (Applied Biosystems) is added, the tubes are vortexed and stored at −20° C. Total RNA is extracted from the blood lysates by the 6100 Nucleic Acid PrepStation according to the manufacturer's instructions (Applied Biosystems). Kidney cortex allograft samples are collected into RNAlater (Ambion), stored overnight at +4° C. and then at −80° C. Finally, tissue is homogenized in a, FastPrep FP120 (Savant) and total RNA is isolated using the RNeasy kit (Qiagen). Quality of the RNA is controlled by Agilent's 2100 Bioanalyzer labchips and the quantity is determined spectrophotometrically.

Genechip analysis: In total, 60 samples from peripheral blood meet the quality and quantity (5-10 μg total RNA) criteria for genechip analysis, including 3 types of grafts (allografts (BN-to-Lewis), isografts (Lewis-to-Lewis) and non-life supporting isografts (Lewis-to-Lewis)) and 3 time-points (days 3, 4, 5 post operation for life-supporting allografts and isografts, days 3, 4 for non-life supporting isografts) which gives a total of 8 groups with a minimal group-size of 6 blood RNA samples.

RNA samples are processed, labeled and hybridized to Affymetrix GeneChip® Rat Expression 230A according to manufacturer's protocol. Expression values are calculated by statistical algorithms of Affymetrix Microarray Suite (MAS) version 5. MAS5 normalized data is analyzed by GeneSpring® 6.2 (Silicon Genetics). The values are normalized (per chip: constant value −150) and all the probesets are filtered for raw values (above 80) and flags (present and marginal), removing about half of the 15923 probesets. All the samples are checked for quality features using the Affymetrix Quality features and condition clustering.

The data are analyzed using statistical filtering on fold change between corresponding groups (d3 allografts versus d3 isografts etc.—cutoff: 2 fold) combined with statistical tests between corresponding groups (d3 allografts versus d3 isografts etc.). As test, the non-para-metric Wilcoxon-Mann-Whitney test is used with a p-value cutoff of 0.005 or 0.005 and using the multiple testing corrections (Benjamini and Hochberg False Discovery Rate). The calculated fold changes of the significant differentially expressed genes are reported in Table 2.

TaqMan confirmation: expression of selected identified candidate AR marker genes are confirmed by quantitative real-time RT-PCR with commercial TaqMan® Gene Expression Assays and Universal PCR Master Mix (Applied Biosystems) on a 7900HT Sequence Detection System (Applied Biosystems) according to the manufacturer's standard protocol. The results are reported in Table 3.

In parallel to transcriptomic analysis, equal amounts of serum of 10 individual rats are pooled to provide samples for differential display using two-dimensional gel electrophoresis. 10 μl of pooled serum is diluted with 390 μl of the sample buffer described (Rabilloud T, Electrophoresis 19 (1998), 758-60). Two-dimensional electrophoresis is carried out as described (Hoving et al., Electrophoresis 21 (2000), 2617-21). Five gels are run in parallel for each sample and stained with the fluorescent dye Sypro Ruby (Molecular Probes). Stained gels are digitized with the FLA-3000 fluorescence imager (Fuji) as TIFF files with 16-bit pixel depth. Image analysis is carried out with the Progenesis (Non-Linear Dynamics) software package. Spot intensities (spot volume, the integral of the optical density over the area) are normalized and statistically compared using the Student t-test. Spots that are significantly different (p<0.05 by t-test and >9-fold difference in volume) between allografted animals and the corresponding isograft controls are excised from the gels and the contained protein identified by MALDI-MS and MS/MS using the 4700 Proteomic Analyzer (Applied Biosystems). Protein identifications are based on matching of the experimentally obtained mass spectra to the SwissProt and/or GenBank databases, using the Mascot (Matrix Science) software.

TABLE 1
List of 28 genes (with NCBI RefSeq/GenBank accession numbers)
which are differentially expressed in acute rejection conditions compared to non
rejection conditions

| | Rat | Human | | | |
|---|---|---|---|---|---|
| Affymetrix | accession | accession | Gene | | |
| probeset | number | number | symbol | Gene name | Notes |
| 1370964_at | NM_013157 | NM_000050, | ASS | arginosuccinate synthetase | |
| | | NM_054012 | | | |
| 1370892_at | NM_031504 | NM_007293□ | C4A | complement component 4a | |
| 1387818_at | NM_053736 | NM_001225, | CASP11 | caspase 11 | human ortholog: |
| | | NM_033306, | | | Casp4 |
| | | NM_033307 | | | |
| 1369290_at | NM_053960 | NM_000579 | CCR5 | chemokine (C-C motif) receptor 5 | |
| 1386907_at | NM_012949 | NM_001976, | ENO3 | enolase 3, beta | |
| | | NM_053013 | | | |
| 1368332_at | NM_133624 | NM_004120 | GBP2 | guanylate binding protein 2, | |
| | | | | interferon-inducible | |
| 1370491_a_at | NM_017016 | NM_002112 | HDC | histidine decarboxylase | |
| 1368073_at | NM_012591 | NM_002198 | IRF1 | interferon regulatory factor 1 | |
| 1370056_at | NM_020103 | ♦ | Ly6c | Ly6-C antigen gene | |
| 1368501_s_at | NM_019323 | ♦ | MCPT9 | mast cell protease 9 | |
| 1389014_at | NM_177928 | NM_005746, | PBEF | pre-B-cell colony-enhancing | |
| | | NM_182790 | | factor | |
| 1370636_at | NM_153721 | NM_002704 | PPBP | pro-platelet basic protein | |
| 1367786_at | NM_080767 | NM_004159, | PSMB8 | proteosome (prosome, | |
| | | NM_148919 | | macropain) subunit, beta type 8 | |
| 1370186_at | NM_012708 | NM_002800, | PSMB9 | proteosome (prosome, | |
| | | NM_148954 | | macropain) subunit, beta type 9 | |
| 1368835_at | NM_032612 | NM_007315, | STAT1 | signal transducer and activator | |
| 1387354_at | | NM_139266 | | of transcription 1 | |
| 1388149_at | NM_032055 | NM_000593 | TAP1 | transporter 1, ATP-binding | |
| | | | | cassette, sub-family B | |
| | | | | (MDR/TAP) | |
| 1372236_at | BI279079 | ♦ | | Rattus norvegicus transcribed | |
| | | | | sequence, possible gene name: | |
| | | | | Card4* | |
| 1372585_at | BM388445 | ♦ | | Rattus norvegicus transcribed | |
| | | | | sequence | |
| 1372604_at | BI289459 | ♦ | | Rattus norvegicus transcribed | |
| | | | | sequence with weak similarity | |
| | | | | to protein ref: NP_055164.1 | |
| | | | | apolipoprotein L 3; TNF-inducible | |
| | | | | protein CG12-1 (H. sapiens) | |
| 1373670_at | AA799569 | NM_005419 | | Similar to signal transducer and | human ortholog: |
| | | | | activator of transcription 2 | TAT2 |
| | | | | (LOC288774), mRNA | |
| 1373992_at | XM_225907 | ♦ | | Rattus norvegicus similar to | |
| | | | | interferon-inducible GTPase | |
| | | | | (LOC307414), mRNA | |
| 1375796_at | BI300770 | XM_293893□ | | Rattus norvegicus similar to | predicted human |
| | | | | Ac2-233 (LOC303163), possible | ortholog: |
| | | | | gene name: Tgtp/Igtb* | LRG-47-like |
| | | | | | protein mRNA |
| 1376144_at | AA819679 | NM_031458□ | | Rattus norvegicus transcribed | possible gene |
| | | | | sequence with similarity to | name: BAL* |
| | | | | protein RefSeqP: NP_113646 B | |
| | | | | aggressive lymphoma gene (H. sapiens) | |
| 1376151_a_at | AI407953 | ♦ | | Rattus norvegicus transcribed | possible gene |
| | | | | sequence with moderate | name: Gtpi* |
| | | | | similarity to protein sp: P00722 | |
| | | | | (E. coli) BGAL_ECOLI beta-galactosidase | |
| | | | | (lactase) | |
| 1382255_at | BE110785 | ♦ | | Rattus norvegicus cDNA clone | possible gene |
| | | | | UI-R-BJ1-avd-e-09-0-UI 3′, | name: Stat2* |
| | | | | mRNA sequence | |
| 1388235_at | X77816 | ♦ | | Rattus norvegicus VSC-beta1 | possible gene |
| | | | | mRNA for PR-Vbeta1 | name: VSC- |
| | | | | | beta1* |
| 1388401_at | BI296155 | NM_001457□ | | Similar to actin-binding protein | possible gene |
| | | | | homolog ABP-278 | name: Flnb* |
| | | | | (LOC306204), mRNA | |
| 1388574_at | BG670966 | NM_004184□ | | similar to tryptophanyl-tRNA | possible gene |
| | | NM_173701□ | | synthetase (LOC362785). | name: Wars* |
| | | NM_213645□ | | mRNA | |
| | | NM_213646□ | | | |

□speculative human ortholog sequence
♦ human ortholog not yet known
*gene name identified by homology search

TABLE 2
Expression level changes of the 28 AR marker genes in rat blood
of AR renal tx model [Fold changes between AR and control
samples; negative values indicate downregulated genes].

| | | Ratio d3 | Ratio d4 | |
|---|---|---|---|---|
| Affymetrix | Gene | allo/iso | allo/iso | Ratio d5 allo/iso |
| probeset | Symbol | blood | blood | blood |
| 1370964_at | ASS | 1.8 | 3.5 | 1.9 |
| 1370892_at | C4A | 3.4 | 3.3 | 3.5 |
| 1387818_at | CASP11 | 2.4 | 2.4 | 2.8 |
| 1369290_at | CCR5 | 2.8 | 3.0 | 4.6 |
| 1386907_at | ENO3 | −2.1 | −2.5 | −2.5 |
| 1368332_at | GBP2 | 9.2 | 6.0 | 4.7 |
| 1370491_a_at | HDC | 4.5 | 7.2 | 4.4 |
| 1368073_at | IRF1 | 4.1 | 3.7 | 2.5 |
| 1370056_at | Ly6c | 2.1 | 2.1 | 1.9 |
| 1368501_s_at | MCPT9 | 4.3 | 31.6 | 8.4 |
| 1389014_at | PBEF | 7.4 | 5.3 | 2.9 |
| 1370636_at | PPBP | 1.4 | −2.3 | −1.9 |
| 1367786_at | PSMB8 | 1.8 | 2.1 | 1.7 |
| 1370186_at | PSMB9 | 1.8 | 2.8 | 1.8 |
| 1368835_at | STAT1 | 3.0/3.2 | 3.0/2.6 | 1.95/1.91 |
| 1387354_at | | | | |
| 1388149_at | TAP1 | 1.9 | 2.7 | 1.9 |
| 1372236_at | | 3.1 | 3.1 | 2.2 |
| 1372585_at | | 2.5 | 2.6 | 1.7 |
| 1372604_at | | 3.9 | 2.8 | 2.2 |
| 1373670_at | | 3.9 | 3.0 | 2.3 |
| 1373992_at | | 2.7 | 3.7 | 2.1 |
| 1375796_at | | 3.8 | 2.8 | 2.4 |
| 1376144_at | | 2.2 | 2.0 | 1.8 |
| 1376151_a_at | | 5.4 | 5.0 | 2.7 |
| 1382255_at | | 3.1 | 3.2 | 2.0 |
| 1388235_at | | −3.2 | −2.6 | −2.4 |
| 1388401_at | | −2.0 | −3.1 | −2.8 |
| 1388574_at | | 4.3 | 3.5 | 2.5 |

TABLE 3
TaqMan confirmation of blood AR marker expression by quantitative RT-PCR.
[fold changes between AR and control samples]

| | day 3 | | day 4 | | day 5 | | |
|---|---|---|---|---|---|---|---|
| Gene | Affymetrix | TaqMan | Affymetrix | TaqMan | Affymetrix | TaqMan | Confirmation |
| Mcpt9 | 4.3 | 2.0 | 31.6 | 13.4 | 8.4 | 12.2 | ✓ |
| Hdc | 4.5 | 3.8 | 7.2 | 19.1 | 4.4 | 8.4 | ✓ |
| Gbp2 | 9.2 | 5.2 | 6.0 | 7.6 | 4.7 | 4.4 | ✓ |
| CCR5 | 2.8 | 1.6 | 3.0 | 4.8 | 4.6 | 4.3 | ✓ |
| Pbef | 7.4 | 4.1 | 5.3 | 5.9 | 2.9 | 2.9 | ✓ |
| Irf1 | 4.1 | 2.7 | 3.7 | 4.1 | 2.5 | 2.2 | ✓ |
| Psmb8 | 1.8 | 1.7 | 2.1 | 3.3 | 1.7 | 2.9 | ✓ |
| Tap1 | 1.9 | 1.2 | 2.7 | 2.7 | 1.9 | 2.7 | ✓ |
| Psmb9 | 1.8 | 1.9 | 2.8 | 3.5 | 1.8 | 2.6 | ✓ |
| Ass | 1.8 | 1.7 | 3.5 | 4.6 | 1.9 | 1.8 | ✓ |
| Ly6c | 2.1 | 1.3 | 2.1 | 2.6 | 1.9 | 1.4 | ✓ |
| Stat1 | 3.0 | 1.2 | 3.0 | 2.5 | 1.9 | 1.1 | (✓) |
| Ppbp | 1.4 | 0.8 | −2.3 | 0.6 | −1.9 | 0.8 | ✓ |

TaqMan data: mean fold change in allograft blood at day x (n = 6) versus isograft blood at day 4 (n = 3)

Quantitative TaqMan experiments with peripheral blood of allo- and syngrafted rats from the BN-to-Lewis renal transplantation study are performed for the known genes with commercially available TaqMan® Gene Expression Assays (Applied Biosystems). Relative quantitation is applied by normalizing to 18S rRNA. In general, all obtained values are corresponding well with the Affymetrix genechip data by analyzing randomly selected 6 blood samples per group and comparing all to mean values of 3 isograft blood samples of day 4.

TABLE 4
Differentially expressed proteins in serum of rats with and without AR of renal grafts

| | Reference | | | | | | |
|---|---|---|---|---|---|---|---|
| | spot | Accession number | | | Isoform | | Fold change |
| | number | SwissProt | GenBank | Protein ID | (app. pl)¹ | P-value² | (allo-/isograft)³ |
| 1 | 1743 | P04276 | J05148 | Vitamin D-binding protein | | 0.002 | 4.2 |
| | | | | precursor | | | |
| 2 | 1991 | P81827 | AA945585 | urinary protein 1 precursor | A (4.6) | 3.0 × 1.0⁻⁵ | 27 |
| | 1993 | | | (RUP-1) | B (4.3) | 0.020 | 55 |
| | 1994 | | | | C (5.0) | 2.0 × 10⁻⁵ | 33 |
| 3 | 1109 | P26644 | X15551 | Beta-2-glycoprotein I | | 0.003 | de novo in allograft |
| | | | | precursor (apo H) | | | |
| 4 | 1374 | P55159 | U94856 | Paraoxonase 1 | A (4.3) | 0.027 | −2.0 |
| | 1376 | | | | B (4.4) | 0.007 | −2.3 |

¹In case of multiple isoforms, the apparent pl of the respective isoform is given with an arbitrary designation;
²P-value (day 5 samples) by t-test;
³Values determined on day 5 samples, positive numbers indicate upregulation in allograft samples, negative numbers downregulation, de novo indicates spots only found in one group.

1. A method of early diagnosing acute allograft rejection (AR) in a transplanted subject comprising
a) taking as a baseline value the level of mRNA expression corresponding to or protein encoded by at least one gene in a body fluid, e.g. in peripheral blood, the gene originating from a preclinical AR model;
b) detecting a level of mRNA expression corresponding to or protein encoded by the at least one gene in a body fluid, e.g. in peripheral blood, of a transplant patient on a regular basis, e.g. every 4 weeks;
c) comparing the first value with the second value, wherein a first value lower or higher than the second value predicts that the transplanted subject is developing AR.

2. A method for monitoring AR in a transplanted subject at risk of developing AR comprising:
a) obtaining a pre-administration sample from a transplanted subject prior to administration of the agent,
b) detecting the level of expression of mRNA corresponding to or protein encoded by the at least one gene in the pre-administration sample,
c) obtaining one or more post-administration samples from the transplanted patient,
d) detecting the level of expression of mRNA corresponding to or protein encoded by the at least one gene in the post-administration sample or samples,
e) comparing the level of expression of mRNA or protein encoded by the at least one gene in the pre-administration sample with the level of expression of mRNA or protein encoded by the at least one gene in the post-administration sample or samples, and
f) adjusting the agent accordingly; the gene originating from a preclinical AR model.

3. A method for preventing, inhibiting, reducing or treating AR in a transplant subject in need of such treatment comprising administering to the subject a compound that modulates the synthesis, expression or activity of one or more genes or gene products, the gene originating from a preclinical AR model, so that at least one symptom of AR is ameliorated.

4. A compound which modulates the synthesis, expression of activity of one or more genes or gene products, the gene originating from a preclinical AR model, for use as a medicament, e.g. for the prevention or treatment of AR in a transplanted subject.

5. A compound which modulates the synthesis, expression of activity of one or more genes or gene products, the gene originating from a preclinical AR model, for prevention or treatment of AR in a transplanted subject.

6-7. (canceled)

8. A method to claim 1, wherein the transplanted subject is a kidney, or heart, or liver, or lung, or bowel transplanted subject.

9. A method according to claim 1, wherein the genes are selected from the group of genes as identified in Table 1.

10. A method according to claim 1, wherein the level of expression of the gene expression is assessed by detecting the presence of a protein corresponding to the gene expression product.

11. A method according to claim 1, wherein the presence of the protein is detected using a reagent which specifically binds to the protein.

12. A method according to claim 1, wherein the level of mRNA expression of one or more genes is detected by techniques selected from the group consisting of Northern blot analysis, reverse transcription PCR and real time quantitative PCR.

13. A method according to claim 1, wherein the level of mRNA expression of a set of genes is detected.

14. (canceled)

15. A method according to claim 2, wherein the transplanted subject is a kidney, or heart, or liver, or lung, or bowel transplanted subject.

16. A method according to claim 3, wherein the transplanted subject is a kidney, or heart, or liver, or lung, or bowel transplanted subject.

17. A method according to claim 2, wherein the genes are selected from the group of genes as identified in Table 1.

18. A method according to claim 3, wherein the genes are selected from the group of genes as identified in Table 1.

19. A method according to claim 2, wherein the level of expression of the gene expression is assessed by detecting the presence of a protein corresponding to the gene expression product.

20. A method according to claim 3, wherein the level of expression of the gene expression is assessed by detecting the presence of a protein corresponding to the gene expression product.

21. A method according to claim 2, wherein the presence of the protein is detected using a reagent which specifically binds to the protein.

22. A method according to claim 3, wherein the presence of the protein is detected using a reagent which specifically binds to the protein.

23. A method according to claim 2, wherein the level of mRNA expression of one or more genes is detected by techniques selected from the group consisting of Northern blot analysis, reverse transcription PCR and real time quantitative PCR.

24. A method according to claim 3, wherein the level of mRNA expression of one or more genes is detected by techniques selected from the group consisting of Northern blot analysis, reverse transcription PCR and real time quantitative PCR.

25. A method according to claim 2, wherein the level of mRNA expression of a set of genes is detected.

26. A method according to claim 3, wherein the level of mRNA expression of a set of genes is detected.
